(19) United States
(12) Patent Application Publication
(10) Pub. No.: US 2020/0069232 A1
(43) Pub. Date: Mar. 5, 2020

(54) LIP FORCE DETECTING APPARATUS

(71) Applicant: SHU-CHEN TSAI, Taipei City (TW)

(72) Inventor: Yoshiaki Akihiro, Taipei City (TW)

(21) Appl. No.: 16/140,678

(22) Filed: Sep. 25, 2018

(30) Foreign Application Priority Data
Aug. 30, 2018 (TW) 107130422

(51) Int. Cl. A61B 5/22; A61B 5/00

(52) U.S. Cl. CPC A61B 5/224; A61B 5/4542; A61B 5/6843; A61B 5/682

(57) ABSTRACT

A method is provided to measure the difference of lip closure force between left and right lips and find weaknesses in left and right lips that are caused by hypoplasia, atrophy and paralysis so that such symptoms can be treated in early stages. A lip force detecting apparatus for detecting the closure force of lips includes a housing, the upper base portion and the lower base portion for lips protruding from the housing and can be pressed using upper and lower lips, the control unit respectively measures the pressing force generated when the left side of lips presses the upper base portion and the lower base portion, and measures the pressing force generated when the right side of lips presses the upper base portion and the lower base portion.

### CROSS-REFERENCE TO RELATED APPLICATIONS

This application claims priority to Taiwanese application No. 107130422 having a filing date of Aug. 30, 2018, the entire contents both of which are hereby incorporated by reference.

### FIELD OF TECHNOLOGY

This following relates to a lip force detecting apparatus that measures the lip closure force of the subject.

### BACKGROUND

In the past, in order to evaluate the effectiveness of lip closure force training using a lips training device, a simple method of using a lip force detecting apparatus for measuring lip closure force is well known.

A lip detecting device that measures the subject's lip force helps health care workers to understand the subject's treatment condition. The device comprises an airbag, a pressing force detecting unit, and a micro control module. The airbag allows the subject to place it between the subject's lips. The pressing force detecting unit is connected to the airbag for detecting the pressing force that the subject exerts on the airbag, and later is generated into a signal. The micro control module is electrically connected to the pressing force detecting unit and can transform the signal into a lip force value.

However, the traditional lip force detecting apparatus cannot measure the force difference between the left and right lips. Therefore, it cannot diagnose symptoms such as hypoplasia, atrophy and paralysis which are caused by cerebral infarction and would lead to difference in left and right lip closure force. Moreover, the traditional apparatus cannot diagnose important issues such as hypoplasia, atrophy and paralysis early.

### SUMMARY

An aspect relates to a lip force detecting apparatus which measures the difference of lip closure force between the left and right lips through a simple method and find weak spots in left and right lips that are caused by hypoplasia, atrophy and paralysis so that such symptoms can be treated early.

The lip force detecting apparatus of embodiments of this invention, a lip force detecting apparatus for detecting the closure force of lips, comprising: a housing; the upper base portion and the lower base portion for lips protruding from said housing and can be pressed using upper and lower lips; the control unit respectively measures the pressing force generated when the left side of lips presses the upper base portion and the lower base portion, and measures the pressing force generated when the right side of lips presses the upper base portion and the lower base portion.

Moreover, the lip force detecting apparatus of embodiments of this invention also comprises a transmitting unit, wherein said detecting unit measures the pressing force generated when the left side of lips presses said base portion for lips and the pressing force generated when the right side of lips presses said base portion for lips, compares the difference of forces between both lips and transmit such signals.

### BRIEF DESCRIPTION

Some of the embodiments will be described in detail, with references to the following Figures, wherein like designations denote like members, wherein:

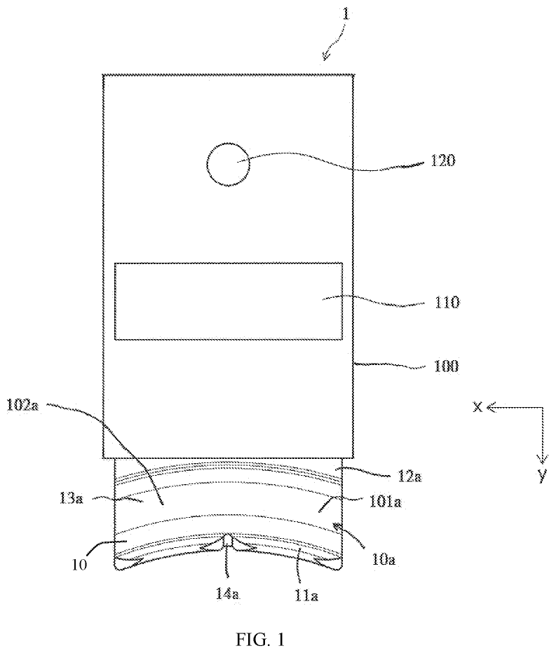

FIG. 1 is a plan view of the lip force detecting apparatus in accordance with a first embodiment of the present invention;

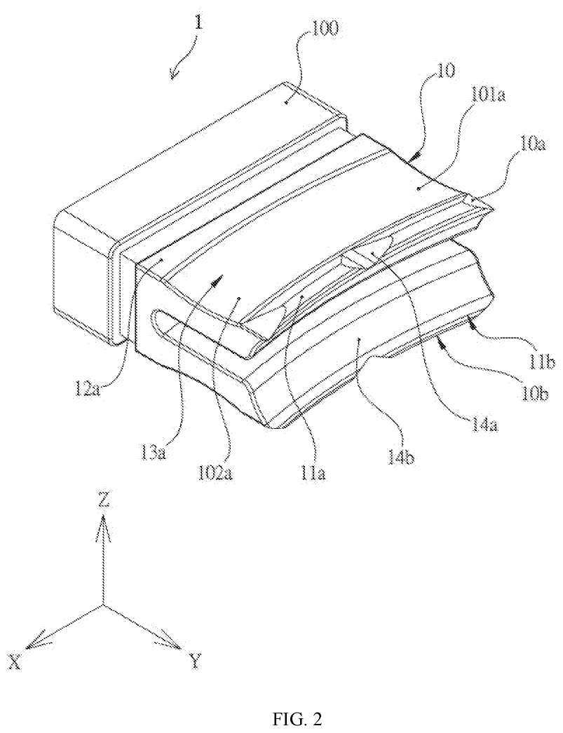

FIG. 2 is a perspective view of a base portion for lips from a front upper diagonal side in accordance with the first embodiment of the present invention;

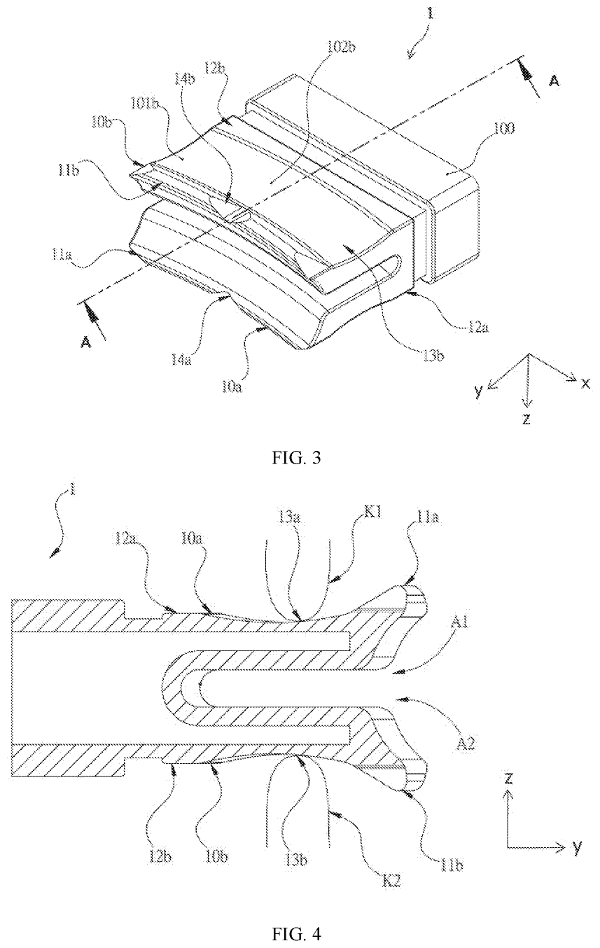

FIG. 3 is a perspective view of a base portion for lips from a front lower diagonal side in accordance with the first embodiment of the present invention;

FIG. 4 is a cross-sectional view of taken along line A-A of FIG. 3;

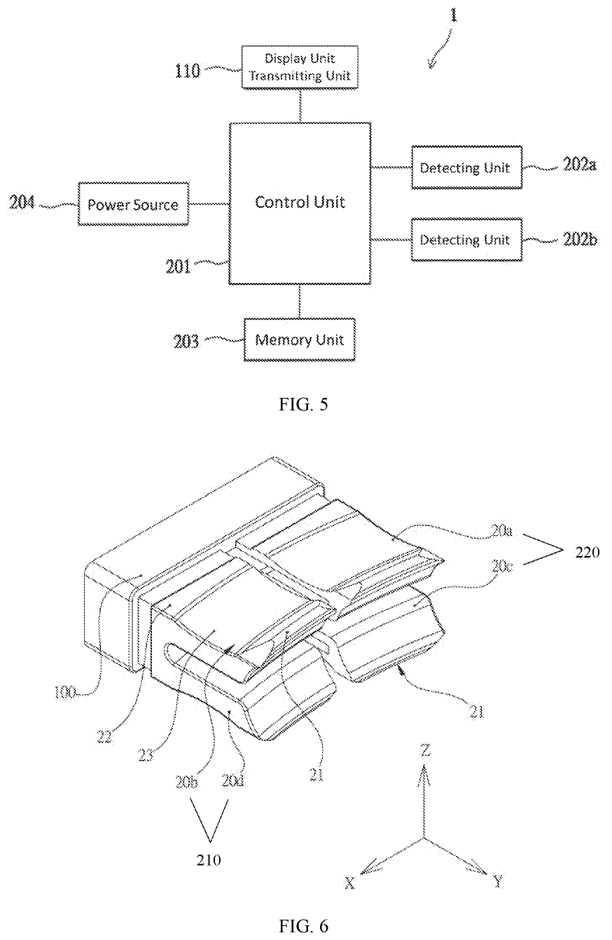

FIG. 5 is a configuration block diagram of the lip force detecting apparatus in accordance with the first embodiment of the present invention;

FIG. 6 is a plan view of the lip force detecting apparatus in accordance with a second embodiment of the present invention;

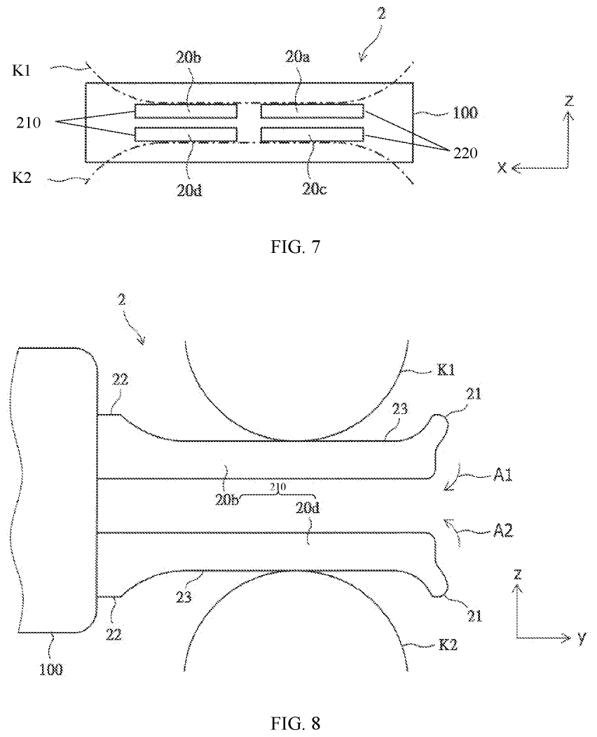

FIG. 7 is the front view of the lip force detecting apparatus in accordance with the second embodiment of the present invention; and

FIG. 8 is the profile view of the lip force detecting apparatus in accordance with the second embodiment of the present invention.

### DETAILED DESCRIPTION

The embodiments below refer to the corresponding figures and describe the lip force detecting apparatus of embodiments of the present invention in detail. Figures are illustrated with description using orthogonal coordinate system of X axis, Y axis and Z axis. The positive direction of Y axis is forward, while the negative direction of Y axis is backward; the direction of X axis is bilateral direction (left-right direction); and the positive direction of Z axis is upward and negative direction of Z axis is downward.

### Embodiment 1

### [Configuration of the Lip Force Detecting Apparatus]

Referring to FIGS. 1 to 5, the lip force detecting apparatus 1 in embodiment 1 of the present invention is described below.

The lip force detecting apparatus 1, comprising, a housing 100, an upper base portion 10a, a lower base portion 10b, a control unit 201, a detecting unit 202a, a detecting unit 202b, a memory unit 203, and a power source 204. The control unit 201, detecting unit 202a, detecting unit 202b, memory unit 203, and power source 204 are stored in the housing 100. Moreover, the control unit 201 and memory unit 203 are composed of the electrical components from the base plate which are omitted in the drawing of housing 100. The upper base portion 10a and the lower base portion 10b form the base portion for lips.

The housing 100 is rectangular and comprises a display unit 110 of the transmitting unit that displays the result of detection of lip closure force, and a power switch 120 for starting the lip force detecting apparatus 1 when in operation. Moreover, the shape of the housing 100 is not limited to rectangular and can be a cylinder or any other shapes.

The base portion for lips 10, wherein the design and type of the base for receiving lips are not limited to linear design and can be in U-shaped design.

The upper base portion 10a protrudes forward from the front side of the housing 100 and has a denting towards the rear end in the middle of the bilateral direction (left-right direction) from the top view, which forms in a curved shape from top view. The upper base portion 10a has a space in between the lower base portion 10b in vertical direction, which is located above the lower base portion 10b. The upper base portion 10a is integrally formed with elastic synthetic resin and can elastically reform. The synthetic resin used to form the upper base portion 10a can be, for example, styrene elastomer, polyester elastomer, silicon rubber, polyethylene or vinyl chloride.

The upper base portion 10a where its upper side of the surface that comes in contact with the lips comprises a front wall portion 11a, a rear wall portion 12a, and a concave surface 13a. The front wall portion 11a is positioned above the upper base portion 10a at the front end and extends upwards from the left end to the right end. There is a notch portion 14a in the center of the extending direction. The rear wall portion 12a is positioned above the upper base portion 10a at the rear end and extends upwards from the left end to the right end. The concave surface 13a is located in the middle of the front wall portion 11a and the rear wall portion 12a, thus forming into a gutter shape in the front and back direction.

The lower base portion 10b protrudes forward from the front side of the housing 100 and has a denting towards the rear end in the middle of the bilateral direction (left-right direction) from the top view, which forms in a curved shape from bottom view. The lower base portion 10b has a space in between the upper base portion 10a in vertical direction, which is located below the upper base portion 10a. The lower base portion 10b is integrally formed with elastic synthetic resin and can elastically reform. The synthetic resin used to form the lower base portion 10b can be for example, styrene elastomer, polyester elastomer, silicon rubber, polyethylene or vinyl chloride.

The lower base portion 10b where its lower side of the surface that comes in contact with the lips comprises a front wall portion 11b, a rear wall portion 12b, and a concave surface 13b. The front wall portion 11b is positioned below the lower base portion 10b at the front end and extends downwards from the left end to the right end. There is a notch portion 14b in the center of the extending direction. The rear wall portion 12b is positioned below the lower base portion 10b at the rear end and extends downwards from the left end to the right end. The concave surface 13b is located in the middle of the front wall portion 11b and the rear wall portion 12b, thus forming into a gutter shape in the front and back direction.

The control unit 201 is activated after the power source 204 supplies power and said control unit 201 processes different tasks through operating the control programs stored in the memory unit 203. Based on the signals input through detecting unit 202a and 202b, the control unit 201 respectively measures the pressing force generated when the left side of lips presses the upper base portion 10a and the lower base portion 10b, and measures the pressing force generated when the right side of lips presses the upper base portion 10a and the lower base portion 10b. According to the pressing force obtained above, the control unit 201 can individually measure the lip closure force of the left and right lips and display the difference of measured lip closure force between the left and right lips on display unit 110 and then transmit such signals.

The detecting unit 202a measures the pressing force applied to the right side portion 101a located at the right side of the notch portion 14a that is visible from the front of the concave surface 13a of the upper base portion 10a, and measures the pressing force applied to the right side portion 101b located at the right side of the notch portion 14b that is visible from the front of the concave surface 13b of the lower base portion 10b, then sends the electric signals of the corresponding measured values to the control unit 101. The detecting unit 202a, for example may be a known loading sensor, detects the magnitude of pressing force applied to the right-side portion 101a of the upper base portion 10a and the right-side portion 101b of the lower base portion 10b. Moreover, the detecting unit 202a may also use sensors other than the loading sensor to measure the pressing force applied to the right-side portion 101a and the right-side portion 101b.

The detecting unit 202b measures the pressing force applied to the left side portion 102a located at the left side of the notch portion 14a that is visible from the front of the concave surface 13a of the upper base portion 10a, and measures the pressing force applied to the left side portion 102b located at the left side of the notch portion 14b that is visible from the front of the concave surface 13b of the lower base portion 10b, then sends the electric signals of the corresponding measured values to the control unit 101. The detecting unit 202b, for example may be a known loading sensor, detects the magnitude of pressing force applied to the left side portion 102a of the upper base portion 10a and the left side portion 102b of the lower base portion 10b. Moreover, the detecting unit 202b may also use sensors other than the loading sensor to measure the pressing force applied to the left side portion 102a and the left side portion 102b.

The memory unit 203 may be control programs that are stored by the control unit 201 in advance.

The power source 204, wherein it supplies electricity to the control unit 201 when the power switch 120 is ON; and it stops supplying electricity to the control unit 201 when the power switch 120 is OFF.

### [Operation of the Lip Force Detecting Apparatus]

Referring to FIGS. 1 to 5, the lip force detecting apparatus 1 in embodiment 1 of the present invention is described below.

The lip force detecting apparatus 1 may be operated by turning the power switch 120 to ON, and the control unit 201 will be activated after power source 204 supplies electricity thereof.

First, the user holds the housing 100 using the upper base portion 10a and the lower base portion 10b as the front side, then places the upper base portion 10a and the lower base portion 10b inside the mouth.

The user uses his upper lips and lower lips to vertically press the upper base portion 10a and the lower base portion 10b which are placed inside the mouth.

The right side of the upper lip presses on the right-side portion 101a of the upper base portion 10a in downward direction. The left side of the upper lip presses on the left side portion 102a of the upper base portion 10a in downward direction. The right side of the lower lip presses on the right-side portion 101b of the lower base portion 10b in upward direction. The left side of the lower lip presses on the left side portion 102b of the lower base portion 10b in upward direction.

More specifically, referring to FIG. 2 to 4, by increasing the pressing force of the right upper lip K1 exerted to the right side portion 101a of the upper base portion 10a, the surface mucosa of the right upper lip K1 expands in front and back direction at the concave surface 13a due to the pressing force exerted on the right side portion 101a; hence, the thickness of the mucosa of upper lip K1 between the muscle tissue of the right side upper lip K1 and the right side portion 101a decreases, causing the muscle tissue of the right side upper lip K1 to press the thinned mucosa tissue downwards (A1 direction) on the right side portion 101a. Meanwhile, the right-side portion 101a may move independently from the left side portion 102a by positioning a notch portion 14a at the front wall portion 11a of the upper base portion 10a. Therefore, it can move according to the pressing force of the right side upper lip K1.

Moreover, by increasing the pressing force of the left upper lip exerted to the left side portion 102a of the upper base portion 10a, the surface mucosa of the left upper lip K1 expands in front and back direction at the concave surface 13a due to the pressing force exerted on the left side portion 102a; hence, the thickness of the mucosa of upper lip K1 between the muscle tissue of the left side upper lip K1 and the left side portion 102a decreases, causing the muscle tissue of the left side upper lip K1 to press the thinned mucosa tissue downwards on the left side portion 102a. Meanwhile, the left side portion 102a may move independently from the right-side portion 101a by positioning a notch portion 14a at the front wall portion 11a of the upper base portion 10a. Therefore, it can move according to the pressing force of the left side upper lip K1.

Furthermore, referring to FIG. 2 to 4, by increasing the pressing force of the right lower lip K2 exerted to the right side portion 101b of the lower base portion 10b, the surface mucosa of the right lower lip K2 expands in front and back direction at the concave surface 13b due to the pressing force exerted on the right side portion 101b; hence, the thickness of the mucosa of lower lip K2 between the muscle tissue of the right side lower lip K2 and the right side portion 101b decreases, causing the muscle tissue of the right side lower lip K2 to press the thinned mucosa tissue upwards (A2 direction) on the right side portion 101b. Meanwhile, the right-side portion 101b may move independently from the left side portion 102b by positioning a notch portion 14b at the front wall portion 11b of the lower base portion 10b. Therefore, it can move according to the pressing force of the right side lower lip K2.

Moreover, by increasing the pressing force of the left lower lip exerted to the left side portion 102b of the lower base portion 10b, the surface mucosa of the left lower lip K2 expands in front and back direction at the concave surface 13b due to the pressing force exerted on the left side portion 102b; hence, the thickness of the mucosa of lower lip K2 between the muscle tissue of the left side lower lip K2 and the left side portion 102b decreases, causing the muscle tissue of the left side lower lip K2 to press the thinned mucosa tissue upwards on the left side portion 102b. Meanwhile, the left side portion 102b may move independently from the right-side portion 101b by positioning a notch portion 14b at the front wall portion 11b of the lower base portion 10b. Therefore, it can move according to the pressing force of the left side lower lip K2.

Further, the lip mucosa in between the upper lip and the right-side portion 101a and the left side portion 102a of the upper base portion 10a and the lip mucosa in between the lower lip and the right-side portion 101a and the left side portion 102a of the lower base portion 10b may become thinner. Therefore, it can exclude the pressure of muscles other than lip muscle and orbicularis oris muscle and such pressing force exerted on the upper base portion 10a and the lower base portion 10b. Only the pressing force of lip muscle tissue exists alone.

Besides, the concave surface 13a and concave surface 13b are formed into a gutter shape. The upper lip may be guided to the right-side portion 101a and the left side portion 102a by the concave surface 13a, and the lower lip may be guided to the right-side portion 102a and the left side portion 102b by the concave surface 13b. Through the process of using the concave surface 13a and the concave surface 13b to guide the upper and lower lips, the mucosa of the surface of upper and lower lips further expands in front and back direction.

Furthermore, the gutter shape of the concave surface 13a and the concave surface 13b allows the concave surface 13a to hold the upper lip and the concave surface 13b to hold the lower lip; hence, to the right side portion 101a and the left side portion 102a, pressing force from the upper lip may be particularly exerted thereof, while to the right side portion 101b and the left side portion 102a, pressing force from the lower lip may be particularly exerted thereof.

The detecting unit 202a may send out electric signals to the control unit 201 according to the corresponding pressure force of the right side of lip applied to the right-side portion 101a of the upper base portion 10a and the right-side portion 101b of the lower base portion 10b. Moreover, the detecting unit 202b may send out electric signals to the control unit 201 according to the corresponding pressure force of the left side of lip applied to the left side portion 102a of the upper base portion 10a and the left side portion 102b of the lower base portion 10b.

The control unit 201 measures the right lip closure force based on the signal input from the detecting unit 202a, and the left lip closure force based on the signal input from the detecting unit 202b. Then the control unit 201 displays the detected right and left lip closure force on the display unit and transmits such signals. Meanwhile, the control unit 201 can display the difference of right and left lip closure force comparatively on the display unit 110. For example, the control unit 201 can display the lip closure force of the right lip and the lip closure force of the left lip in parallel on the display unit 110 and transmit such signals. The control unit 201 can exclude the pressing forces exerted by muscles other than lips muscle and orbicularis oris muscle and only measures the pure pressing force of lip muscle tissue. It can accurately measure the right and left lip closure force.

The user and health care workers can refer to the right and left lips closure force displayed on the display unit 110 to determine the weak spots of right and left lips. They can also notice hypoplasia, atrophy or paralysis at the weak spot so that such symptoms can be treated at early stage.

According to this embodiment, this invention individually measures the pressing force generated when the left and right lips presses the upper base portion 10a and the lower base portion 10b (base portion for lips 10) respectively. It can calculate the pressing force difference of right and left lip closure force through simple operations, and can discover hypoplasia, atrophy or paralysis at the weak spot and treat these symptoms at early stage.

Although in this embodiment, the front wall portion 11a is located at the front side of the upper base portion 10a and the front wall portion 11b is located at the front side of the lower base portion 10b, the front wall portion 11a can also be placed slightly towards the back of the front side of the upper base portion and the front wall portion 11b can also be placed slightly towards the back of the front side of the lower base portion.

### Embodiment 2

Referring to FIGS. 6 to 8, the lip force detecting apparatus 2 in embodiment 2 of the present invention is described below.

In FIGS. 6 to 8, the configurations in FIGS. 1 to 4 that are the same with the embodiment 2 are labeled with the same code. Hence, their descriptions are omitted.

The lip force detecting apparatus 2, comprising a housing 100, an upper right base portion 20a, an upper left base portion 20b, a lower right base portion 20c, a lower left portion 20d, a control unit 201, a detecting unit 202a, a detecting unit 202b, a memory unit 203, and a power source 204. The upper right base portion 20a, the upper left base portion 20b, the lower base portion 20c and the lower left portion 20d form into the base portion for lips. Moreover, the upper left base portion 20b and the lower left base portion 20d form the first base portion 210; whereas the upper right base portion 20a and the lower right base portion 20c form the second base portion 220.

The housing 100 is rectangular and comprises a display unit 110 of the transmitting unit that displays the result of detection of lip closure force, and a power switch 120 for starting the lip force detecting apparatus 1 when in operation.

The upper right base portion 20a protrudes forward from the front side of the housing 100, which has a space in between the lower right base portion 20c in vertical direction and a space in between the upper left base portion 20b in bilateral (left-right) direction. The upper right base portion 20a where its upper side of the surface that comes in contact with the lips comprises a front wall portion 21, a rear wall portion 22, and a concave surface 23. The front wall portion 21 is positioned above the upper right base portion 20a at the front end and extends upwards from the left end to the right end. The rear wall portion 22 is positioned above the upper right base portion 20a at the rear end and extends upwards from the left end to the right end. The concave surface 23 is located in the middle of the front wall portion 21 and the rear wall portion 22, thus forming into a gutter shape in the front and back direction.

The upper left base portion 20b protrudes forward from the front side of the housing 100, which has a space in between the lower left base portion 20d in vertical direction and a space in between the upper right base portion 20a in bilateral (left-right) direction. The upper left base portion 20b, referring to FIG. 8, where its upper side of the surface that comes in contact with the lips comprises a front wall portion 21, a rear wall portion 22, and a concave surface 23. The front wall portion 21 is positioned above the upper left base portion 20b at the front end and extends upwards from the left end to the right end. The rear wall portion 22 is positioned above the upper left base portion 20b at the rear end and extends upwards from the left end to the right end. The concave surface 23 is located in the middle of the front wall portion 21 and the rear wall portion 22, thus forming into a gutter shape in the front and back direction.

The lower right base portion 20c protrudes forward from the front side of the housing 100, which has a space in between the upper right base portion 20a in vertical direction and a space in between the lower left base portion 20d in bilateral (left-right) direction. The lower right base portion 20c where its lower side of the surface that comes in contact with the lips comprises a front wall portion 21, a rear wall portion 22, and a concave surface 23. The front wall portion 21 is positioned above the lower right base portion 20c at the front end and extends downwards from the left end to the right end. The rear wall portion 22 is positioned below the lower right base portion 20c at the rear end and extends downwards from the left end to the right end. The concave surface 23 is located in the middle of the front wall portion 21 and the rear wall portion 22, thus forming into a gutter shape in the front and back direction.

The lower left base portion 20d protrudes forward from the front side of the housing 100, which has a space in between the upper left base portion 20b in vertical direction and a space in between the lower right base portion 20c in bilateral (left-right) direction. The lower left base portion 20d, referring to FIG. 8, where its lower side of the surface that comes in contact with the lips comprises a front wall portion 21, a rear wall portion 22, and a concave surface 23. The front wall portion 21 is positioned below the lower left base portion 20d at the front end and extends downwards from the left end to the right end. The rear wall portion 22 is positioned below the lower left base portion 20d at the rear end and extends downwards from the left end to the right end. The concave surface 23 is located in the middle of the front wall portion 21 and the rear wall portion 22, thus forming into a gutter shape in the front and back direction.

The detecting unit 202a detects the pressing force of the upper right base portion 20a and the lower right base portion 20c and output the corresponding signal to the control unit 201.

The detecting unit 202b detects the pressing force of the upper left base portion 20b and the lower left base portion 20d and output the corresponding signal to the control unit 201.

### [Operation of the Lip Force Detecting Apparatus]

Referring to FIGS. 6 to 8, the operation of the lip force detecting apparatus 2 in embodiment 2 of the present invention is described below.

The lip force detecting apparatus 2 may be operated by turning the power switch 120 to ON, and the control unit 201 will be activated after power source 204 supplies electricity thereof.

First, the user holds the housing 100 using the upper right base portion 20a, the upper left base portion 20b, the lower right base portion 20c and the lower left base portion 20d as the front side, then places the upper right base portion 20a, the upper left base portion 20b, the lower right base portion 20c and the lower left base portion 20d inside the mouth.

The user uses his upper lips and lower lips to vertically press the upper right base portion 20a, the upper left base portion 20b, the lower right base portion 20c and the lower left base portion 20d which are placed inside the mouth.

The right side of the upper lip presses on the upper right-side portion 20a in downward direction. The left side of the upper lip presses on the upper left side portion 20b in downward direction. The right side of the lower lip presses on the lower right-side portion 20c in upward direction. The left side of the lower lip presses on the lower left side portion 20d in upward direction.

More specifically, by increasing the pressing force of the right upper lip K1 exerted to the concave surface 23 of the upper right side portion 20a, the surface mucosa of the right upper lip K1 expands in front and back direction at the concave surface 23 due to the pressing force exerted on the bottom of the concave surface 23; hence, the thickness of the mucosa of upper lip K1 between the muscle tissue of the right side upper lip K1 and the bottom of the concave surface 23 decreases, causing the muscle tissue of the right side upper lip K1 to press the thinned mucosa tissue downwards on the upper right side portion 20a.

Moreover, referring to FIG. 8, by increasing the pressing force of the left upper lip K1 exerted to the concave surface 23 of the upper left side portion 20b, the surface mucosa of the left upper lip K1 expands in front and back direction at the concave surface 23 due to the pressing force exerted on the bottom of the concave surface 23; hence, the thickness of the mucosa of upper lip K1 between the muscle tissue of the left side upper lip K1 and the bottom of the concave surface 23 decreases, causing the muscle tissue of the left side upper lip K1 to press the thinned mucosa tissue downwards (A1 direction) on the upper left side portion 20b.

Furthermore, by increasing the pressing force of the right lower lip K2 exerted to the concave surface 23 of the lower right side portion 20c, the surface mucosa of the right lower lip K1 expands in front and back direction at the concave surface 23 due to the pressing force exerted on the bottom of the concave surface 23; hence, the thickness of the mucosa of lower lip K2 between the muscle tissue of the right side lower lip K2 and the bottom of the concave surface 23 decreases, causing the muscle tissue of the right side lower lip K2 to press the thinned mucosa tissue upwards on the lower right side portion 20c.

Additionally, by increasing the pressing force of the left lower lip K2 exerted to the concave surface 23 of the lower left side portion 20d, the surface mucosa of the left lower lip K2 expands in front and back direction at the concave surface 23 due to the pressing force exerted on the bottom of the concave surface 23; hence, the thickness of the mucosa of lower lip K2 between the muscle tissue of the left side lower lip K2 and the bottom of the concave surface 23 decreases, causing the muscle tissue of the left side lower lip K2 to press the thinned mucosa tissue upwards on the lower left side portion 20d.

Further, the lip mucosa in between the upper lip and the upper right-side portion 20a and the upper left side portion 20b of the bottom of the concave surface 23 and the lip mucosa in between the lower lip and the lower right-side portion 20c and the lower left side portion 20d may become thinner. Therefore, it can exclude the pressure of muscles other than lip muscle and orbicularis oris muscle and such pressing force exerted on the upper right-side portion 20a, upper left side portion 20b, lower right-side portion 20c and lower left side portion 20d. Only the pressing force of lip muscle tissue exists alone.

Besides, the concave surface 23 is formed into a gutter shape. The upper lip and lower lip may be guided to the concave surface 23 by the bottom of the concave surface 23. Through the process of using the concave surface 23 to guide the upper and lower lips, the mucosa of the surface of upper and lower lips further expands in front and back direction.

Furthermore, the gutter shape of the concave surface 23 allows the concave surface 23 to hold the upper lip and the lower lip; hence, to the bottom of the concave surface 23, pressing force from the upper lip and lower lip may be particularly exerted thereof.

The detecting unit 202a may send out electric signals to the control unit 201 according to the corresponding pressure force of the right side of lip applied to the upper right-side portion 20a and the lower right-side portion 20c. Moreover, the detecting unit 202b may send out electric signals to the control unit 201 according to the corresponding pressure force of the left side of lip applied to the upper left side portion 20b and the lower left side portion 20d.

The control unit 201 measures the right lip closure force based on the signal input from the detecting unit 202a, and the left lip closure force based on the signal input from the detecting unit 202b. Then the control unit 201 displays the detected right and left lip closure force on the display unit 110 and transmits such signals. Meanwhile, the control unit 201 can display the difference of right and left lip closure force comparatively on the display unit 110. For example, the control unit 201 can display the lip closure force of the right lip and the lip closure force of the left lip in parallel on the display unit 110 and transmit such signals. The control unit 201 can exclude the pressing forces exerted by muscles other than lips muscle and orbicularis oris muscle and only measures the pure pressing force of lip muscle tissue. It can accurately measure the right and left lip closure force.

The user and health care workers can refer to the right and left lips closure force displayed on the display unit 110 to determine the weak spots of right and left lips. They can also notice hypoplasia, atrophy or paralysis at the weak spot so that such symptoms can be treated at early stage.

According to this embodiment, this invention individually measures the pressing force generated when the left lip presses the upper right base portion 20a and the lower right base portion 20c (base portion for lips 10), and the right lip presses the upper left base portion 20b and the lower left base portion 20d (base portion for lips 10). It can calculate the pressing force difference of right and left lip closure force through simple operations, and can discover hypoplasia, atrophy or paralysis at the weak spot and treat these symptoms at early stage.

In this embodiment, the upper right base portion 20a and the lower right base portion 20c, the upper left portion 20b and the lower left base portion 20d has a spacing in between and are placed in bilateral (left-right) direction. This enables the upper right base portion 20a and the lower right base portion 20c, the upper left portion 20b and the lower left base portion 20d to move independently and measure the lip closure force of left and right lips more precisely.

In this embodiment, the front wall portion 21 is located at the front side of the upper right base portion 20a, the upper left base portion 20b, the lower right base portion 20c and the lower left base portion 20d. The front wall portion 21 can also be placed slightly towards the back of the front side of the upper right base portion 20a, the upper left base portion 20b, the lower right base portion 20c and the lower left base portion 20d.

In this embodiment, although the front portion of the upper right base portion 20a, the upper left portion 20b, the lower right portion 20c, and the lower left portion 20d are in a linear shape when viewed from the above or below, the front portion of the upper right base portion 20a, the upper left portion 20b, the lower right portion 20c, and the lower left portion 20d can also be in a curved shape that gradually protrudes from the inner side to the outer side towards the front portion when viewed from the above or below.

The types, configuration and quantity of the components in embodiments of this invention are not limited to the aforementioned embodiments. Proper modifications to change the components and achieve the same results are possible within the scope of embodiments of the invention.

More specifically, in said embodiments 1 and 2, although said embodiments 1 and 2 display the right and left lip closure force on the display unit 110 and transmit their signals, said embodiments can also transmit the signal of the right and left lip closure force through speakers in the form of audio or sound. It can also transmit the signal of the right and left lip closure force through LED lights in the form of light.

Moreover, in the first and second embodiment, although the difference of the right and left lip closure force can be comparatively shown by said embodiments on the display unit 110, said embodiments can also store the pressing force detected from the detecting unit 202a and the pressing force detected from the detecting unit 202b in an external memory drive. In this situation, when the external memory drive is connected to an external personal computer, the difference of the right and left lip closure force may be displayed in comparison in the monitor.

The lip force detecting apparatus of embodiments of present invention is used to measure the lip closure force.

Although the present invention has been disclosed in the form of preferred embodiments and variations thereon, it will be understood that numerous additional modifications and variations could be made thereto without departing from the scope of the invention.

For the sake of clarity, it is to be understood that the use of ‘a’ or ‘an’ throughout this application does not exclude a plurality, and ‘comprising’ does not exclude other steps or elements.

What is claimed is:

1. A lip force detecting apparatus for detecting a closure force of lips, comprising:
a housing:
a base portion for lips protruding from the housing, the base portion configured to be pressed using upper and lower lips; and
a detecting unit measuring: i) a pressing force applied to the base portion by pressing a left side of lips, and ii) a pressing force applied to the base portion by pressing a right side of lips.

2. The lip force detecting apparatus of claim 1, wherein the base portion comprises a front wall portion located at an upper side and a lower side that extends horizontally from a left end to a right end, respectively, further wherein the front wall portion has a notch portion in a middle of a horizontal extension.

3. The lip force detecting apparatus of claim 1, wherein the base portion is formed by positioning a first base portion and a second base portion on the left side and the right side with a gap in between, further wherein the detecting unit respectively detects the pressing force generated when the left side of lips presses the first base portion and the pressing force generated when the right side of lips presses the second base portion.

4. The lip force detecting apparatus of claim 1, wherein the base portion comprises a concave surface at a surface where the lip force detecting apparatus contacts the lips.

5. The lip force detecting apparatus of claim 1, comprising: a transmitting unit, wherein the detecting unit measures the pressing force generated when the left side of lips presses the base portion and the pressing force generated when the right side of lips presses the base portion, compares a difference of forces between both lips and transmit such signals.
